(19) United States
(12) Patent Application Publication
(10) Pub. No.: US 2021/0192291 A1
(43) Pub. Date: Jun. 24, 2021

(54) CONTINUOUS TRAINING FOR AI NETWORKS IN ULTRASOUND SCANNERS

(71) Applicant: GE Precision Healthcare LLC, Wauwatosa, WI (US)

(72) Inventors: Kristin Sarah McLeod, Oslo (NO); Svein Arne Aase, Trondheim (NO)

(21) Appl. No.: 16/722,491

(22) Filed: Dec. 20, 2019

(51) Int. Cl. G06K 9/62

(52) U.S. Cl. CPC G06K 9/6263; G06K 9/6254; G06K 9/6256

(57) ABSTRACT

Continuous training of an artificial intelligence (AI) model for an ultrasound scanner is provided. A method for the training comprises generating an image of a target using an AI model, detecting, by a processor, a correction of the target image by an operator. One or both of the following may be saved: the corrected image, and the target image and correction data to the target image. The ultrasound scanner may initiate training the AI model using one of: the corrected image and the target image and correction data for the target image.

### FIELD

Certain embodiments relate to ultrasound imaging. More specifically, certain embodiments relate to continuous training for artificial intelligence (AI) networks in ultrasound scanners.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Artificial intelligence processing of ultrasound images and/or video is often applied to process the images and/or video to assist an ultrasound operator or other medical personnel viewing the processed image data with providing a diagnosis. However, the artificial intelligence (AI) processing of ultrasound images and/or video depends on number of images used in training the AI model. Compared to the potential number of images encountered after deployment, the number of images in training is limited.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

Continuous training for artificial intelligence (AI) networks in ultrasound scanners is disclosed.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1A is a block diagram of an exemplary ultrasound system that is operable to facilitate interaction by an ultrasound operator with an artificial intelligence (AI) processor, in accordance with various embodiments.

FIG. 1B is a block diagram of an exemplary ultrasound system communicating with other electronic devices, in accordance with various embodiments.

FIG. 2 is an exemplary flow diagram for setting up an exemplary ultrasound system for continuous training, in accordance with various embodiments.

FIG. 3 is an exemplary flow diagram for providing corrected images for continuous training, in accordance with various embodiments.

FIG. 4 is an exemplary flow diagram for training the AI processor with the corrected images for continuous training, in accordance with various embodiments.

### DETAILED DESCRIPTION

Certain embodiments may be found for providing continuous training for artificial intelligence (AI) networks inside ultrasound scanners. Various embodiments may have the technical effect of improving AI algorithms on local ultrasound scanners without sending any images to a central server that may be, for example, outside a local network, and receiving training models from the central server. A local network may comprise devices that are behind a common firewall such as, for example, a router, a bridge, etc. Aspects of the present disclosure have the technical effect of local ultrasound scanners improving AI algorithms more often than if a local ultrasound scanner is only receiving AI models trained by a central server.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and where the process does not form any “beams”. Also, forming of images may be performed using channel combinations that may originate from more than one transmit event (e.g., synthetic aperture techniques).

While various descriptions are made with respect to an ultrasound system for the sake of expedience, it should be understood that any embodiment of the disclosure may also be used with other image scanning machines that use artificial intelligence and where the generated images are able to be corrected by the operator.

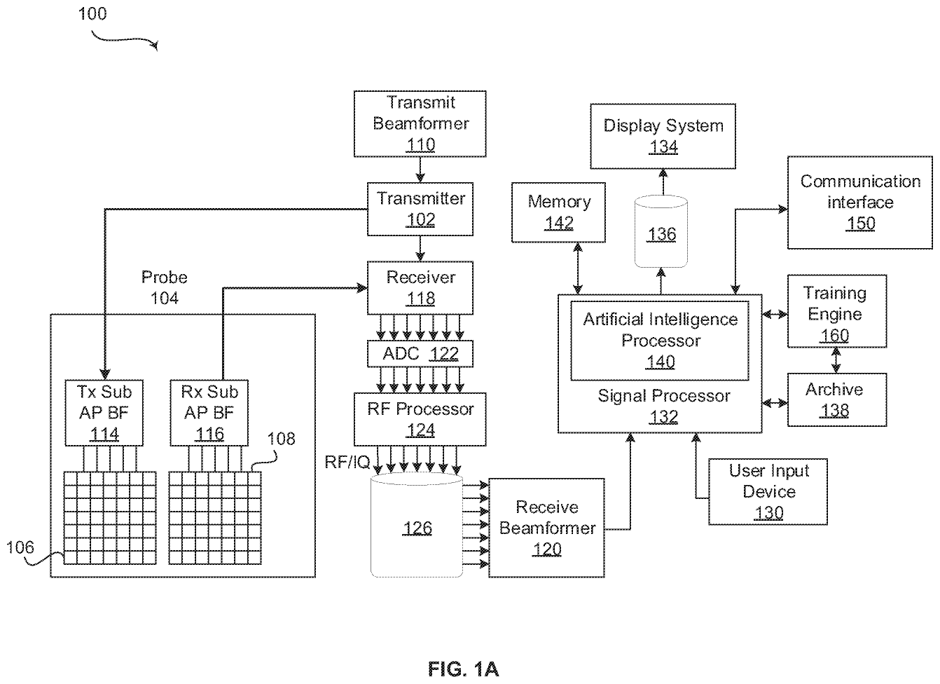

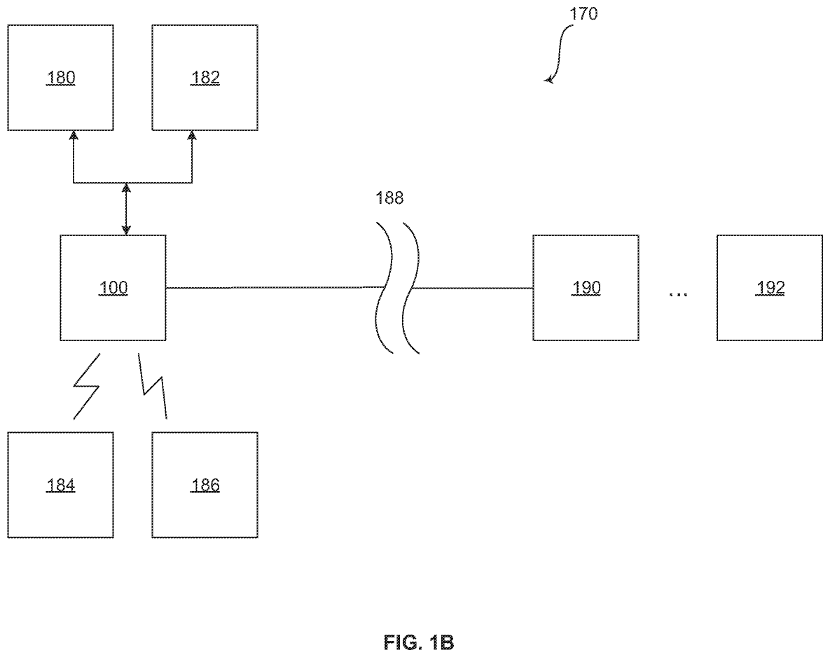

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1A.

FIG. 1A is a block diagram of an exemplary ultrasound system 100 that is operable to facilitate interaction by an ultrasound operator with an artificial intelligence (AI) processor 140 configured to, for example, classify, landmark detect, segment, annotate, identify, and/or track biological and/or artificial structures in ultrasound images, in accordance with various embodiments. Referring to FIG. 1A, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, an archive 138, memory 142, a communication interface 150, and a training engine 160.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, interact with an artificial intelligence processor 140 to select tracking targets, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display. As another example, user input device 130 may include an accelerometer, gyroscope, and/or magnetometer attached to and/or integrated with the probe 104 to provide gesture motion recognition of the probe 104, such as to identify one or more probe compressions against a patient body, a pre-defined probe movement or tilt operation, or the like. Additionally and/or alternatively, the user input device 130 may include image analysis processing to identify probe gestures by analyzing acquired image data.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an artificial intelligence processor 140 and may be capable of receiving input information from a user input device 130 and/or archive 138, generating an output displayable by a display system 134, and manipulating the output in response to input information from a user input device 130, among other things. The signal processor 132 and artificial intelligence processor 140 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an artificial intelligence processor 140 that comprises suitable logic, circuitry, interfaces and/or code that may be operable to analyze acquired ultrasound images to classify, landmark detect, segment, annotate, identify, and/or track biological and/or artificial structures in ultrasound images. The biological structures may include, for example, nerves, vessels, organ, tissue, or any suitable biological structures. The artificial structures may include, for example, a needle, an implantable device, or any suitable artificial structures. The artificial intelligence processor 140 may include, for example, one or more of the following: artificial intelligence image analysis algorithms, one or more deep neural networks (e.g., a convolutional neural network), and/or may utilize any suitable form of artificial intelligence image analysis techniques or machine learning processing functionality configured to analyze acquired ultrasound images to classify, landmark detect, segment, annotate, identify, and/or track biological and/or artificial structures in ultrasound images.

The artificial intelligence processor 140 may comprise suitable logic, circuitry, interfaces and/or code that may be operable to analyze acquired ultrasound images to classify, landmark detect, segment, annotate, identify, and/or track biological and/or artificial structures in ultrasound images. For example, classification may comprise determining a specific class an image or volume may belong to. Landmark detection may comprise determining where in an image or volume a specific structure or point may be. Segmentation may be determining a boundary between two structures.

In various embodiments, the artificial intelligence processor 140 may be provided as a deep neural network that may be made up of, for example, an input layer, an output layer, and one or more hidden layers in between the input and output layers. Each of the layers may be made up of a plurality of processing nodes that may be referred to as neurons. For example, the artificial intelligence processor 140 may include an input layer having a neuron for each pixel or a group of pixels from a scan plane of an anatomical structure. The output layer may have a neuron corresponding to a plurality of pre-defined biological and/or artificial structures. As an example, if performing an ultrasound-based regional anesthesia procedure, the output layer may include neurons for a brachial plexus nerve bundle, the axillary artery, beveled regions on anesthetic needles, and the like. If performing a cardiac related procedure, the output layer may include neurons for “valves,” “ventricles,” “ventricle walls,” “atria,” “outflow tract,” “aorta,” “apex,” “myocardium,” “endocardial border,” “pericardium,” etc.

Other ultrasound procedures may utilize output layers that include neurons for nerves, vessels, bones, organs, needles, implantable devices, or any suitable biological and/or artificial structure. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the artificial intelligence processor 140 deep neural network (e.g., convolutional neural network) may identify biological and/or artificial structures in ultrasound image data with a high degree of probability.

In certain embodiments, the artificial intelligence processor 140 may be configured to identify biological and/or artificial structures based on a user instruction via the user input device 130. For example, the artificial intelligence processor 140 may be configured to interact with a user via the user input device 130 to receive instructions for searching the ultrasound image. As an example, a user may provide a voice command, probe gesture, button depression, or the like that instructs the artificial intelligence processor 140 to search for a particular structure and/or to search a particular region of the ultrasound image.

While an embodiment of the disclosure described the signal processor 132 as including the artificial intelligence processor 140, various embodiments of the disclosure need not be limited so. For example, the artificial intelligence processor 140 may be a separate processor, or part of another processor than the signal processor 132. In some embodiments, the artificial intelligence processor 140 may comprise one or more software modules executed by a processor such as, for example, the RF processor 124 and/or the signal processor 132.

The memory 142 may comprise volatile memory, non-volatile memory, storage devices, etc., that may be used by various devices in the ultrasound system 100. For example, there may be an application that can be downloaded to the memory 142, and used when necessary. The memory 142 may also hold various data that may be used by one or more devices such as, for example, the RF processor 124, the signal processor 132, the artificial intelligence processor 140, etc.

Still referring to FIG. 1A, the training engine 160 may comprise suitable logic, circuitry, interfaces and/or code that may be operable to train the neurons of the deep neural network(s) of the artificial intelligence segmentation module 140. For example, the artificial intelligence processor 140 may be trained to automatically identify and segment biological and/or artificial structures provided in an ultrasound scan plane. For example, the training engine 160 may train, for example, the deep neural networks of the artificial intelligence processor 140 using databases(s) of classified ultrasound images of various structures.

As an example, the artificial intelligence processor 140 may be trained by the training engine 160 with ultrasound images of particular biological and/or artificial structures to train the artificial intelligence processor 140 with respect to the characteristics of the particular structure, such as the appearance of structure edges, the appearance of structure shapes based on the edges, the positions of the shapes relative to landmarks in the ultrasound image data, and the like. In an exemplary embodiment, the structures may include a brachial plexus nerve bundle, the axillary artery, beveled regions on anesthetic needles, and/or any suitable organ, nerve, vessel, tissue, needle, implantable device, or the like. The structural information may include information regarding the edges, shapes, and positions of organs, nerves, vessels, tissue, needles, implantable devices, and/or the like. In various embodiments, the databases of training images may be stored in the archive 138 or any suitable data storage medium.

Accordingly, the artificial intelligence processor 140 may comprise suitable logic, circuitry, interfaces, and/or code that may be operable to label the identified biological and/or artificial structures. For example, the artificial intelligence processor 140 may label the identified structures identified by the output layer of the deep neural network.

In certain embodiments, ultrasound system 100 may communicate via wired or wireless communication with external devices such as, for example, other ultrasound systems, various other types of medical devices including, various network devices such as, for example, personal computers, laptop computers, etc., used by medical personnel, etc.

For example, the training engine 160 and/or the training image databases may be external system(s) communicatively coupled via a wired or wireless connection to the ultrasound system 100 using the communication interface 150. The communication interface may support, for example, one or more wired interfaces (ETHERNET, USB, FIREWIRE, etc.) and/or one or more wireless interfaces (cellular, WIFI, etc.).

FIG. 1B is a block diagram of an exemplary ultrasound system communicating with other electronic devices, in accordance with various embodiments. Referring to FIG. 1B, there is shown a network 170 comprising the ultrasound system 100 communicating via its communication interface 150 by wire to electronic device 180 and 182, and wirelessly to the electronic devices 184 and 186. The electronic devices 180-186 may be, for example, other ultrasound systems, medical equipment, computers of various types used by medical personnel, etc. The ultrasound system 100 and the electronic devices 180-186 may be part of, for example, a local network.

The network 170 may also comprise the ultrasound system 100 communicating via its communication interface 150 with electronic devices 190 . . . 192 through, for example, a communication gateway 188. The ultrasound system 100 may communicate via wire or wirelessly with the communication gateway 188. Some of the electronic devices 190 . . . 192 may be in a same wide area network (WAN) as the ultrasound system 100 and others of the electronic devices 190 . . . 192 may not be a part of the WAN. For example, the electronic device 190 may be in a same WAN as the ultrasound system 100, and the electronic device 192 may be a central server that is not a part of the same WAN, and is farther away from the ultrasound system 100 than the electronic device 190. The central server 192 may, for example, provide information, training updates, etc. for a plurality of widely spread out ultrasound systems.

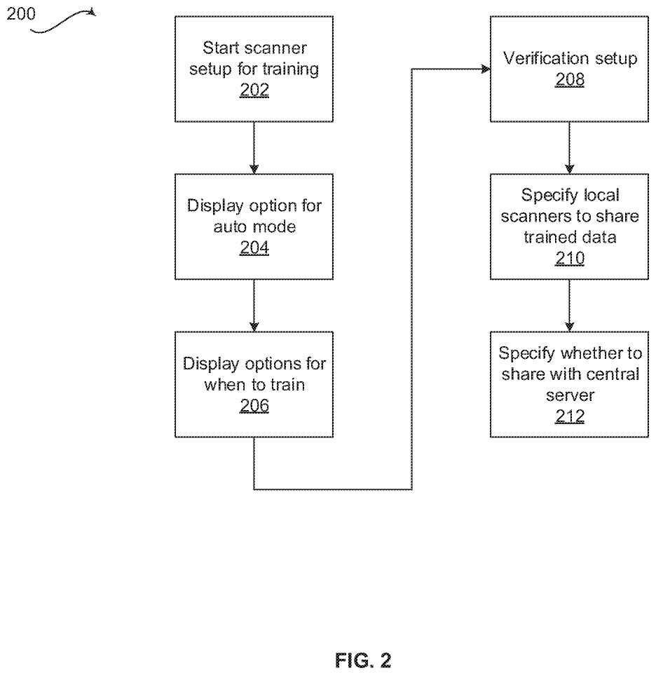

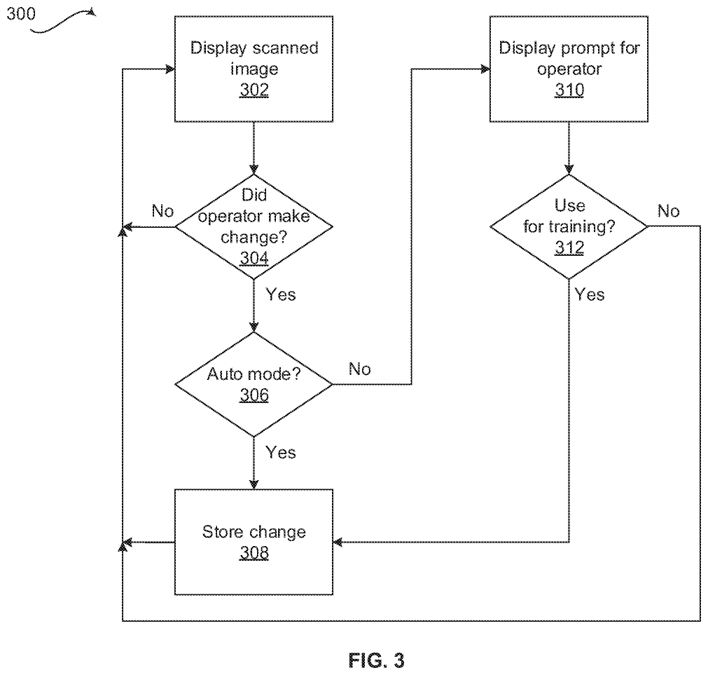

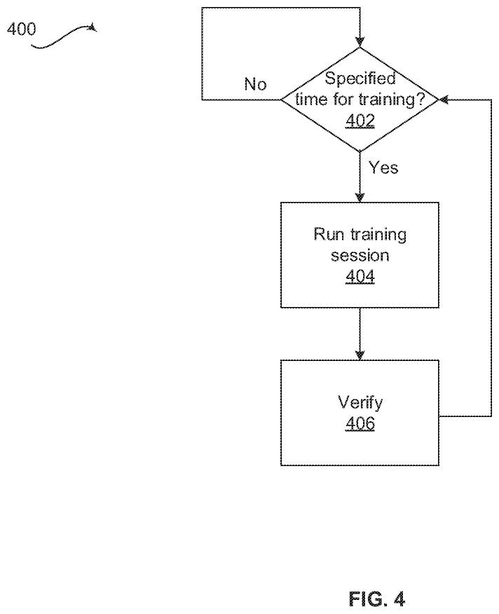

FIG. 2 is an exemplary flow diagram for setting up an exemplary ultrasound system for continuous training, in accordance with various embodiments. Referring to FIG. 2, there is shown the flow diagram 200 comprising blocks 202 to 212. In block 202, the ultrasound system 100 may be set up for continuous training. Continuous training is described in more detail below and with respect to FIGS. 3 and 4. The continuous training setup may be conducted via, for example, the display system 134 and the user input device 130. It may be noted that the display system 134 may comprise a touchscreen as a user input device 130.

A user (an operator) may initiate the continuous training setup, for example, at any time by entering an appropriate command, selecting a menu item, pressing a dedicated button, etc. The ultrasound system 100 may also, for example, display an option via the display system 134 for the continuous training setup upon a first power-up of the ultrasound system 100.

At block 204, there may be displayed on the display system 134 whether to select auto mode for continuous training. The auto mode may save all images that are corrected by the operator for continuous training. If the auto mode is not selected, then the operator may need to enter whether each specific, corrected image should be used for continuous training. This may be in response to, for example, a displayed prompt when the operator corrects the image.

A recorded image comprising annotations, segmentations, classification, etc., may also be referred to as a “target image.” The recorded image may be processed by the AI processor 140 to provide annotations, segmentations, classification, etc. A “corrected image” may refer to a target image to which corrections are made. While there may be some corrections to the image, it should be understood that “corrected image” also refers to correction of the classification of the image, correction to the segmentation/landmark detection on the image, changes to the annotation of the image, etc. Accordingly, a “corrected image” may refer to changes to the image data, changes to the metadata of the image, and/or changes to other data associated with the image.

The images may be generated using the initially trained AI processor 140. The initial training of the AI processor 140 may occur at a factory before shipping the ultrasound system 100. Some embodiments may have auto mode and manual mode, and selecting one mode may disable the other.

The operator may examine the image generated by the ultrasound system 100 using the AI processor 140 and make corrections using, for example, a caliper on the display system 134. The corrections may be, for example, to the annotations, segmentations, classification, etc. of a recorded image. In auto mode, all corrected images may be saved. For example, in an embodiment, the ultrasound system 100 may save a copy of the original image and/or the corrected image in the archive 138. The ultrasound system 100 may then save an anonymized copy of the corrected image in the archive 138 where all patient information may be removed from the anonymized copy of the image. Some embodiments may also remove time information from the anonymized copy of an image.

Some embodiments of the disclosure may anonymize the original image and save the anonymized image and the corrections as correction data (or reference data). Accordingly, the anonymized original image may need to be processed to take into account the corrections when training takes place. For the sake of simplicity, the training process will be presumed to use the anonymized corrected image.

At block 206, various options may be selected for when to start training the AI processor 140 with the new corrected image(s). For example, there may be an option to start training after a certain number of corrected images are saved. This may be, for example, any number of corrected images from one image to a higher number of images.

There may also be an option of starting the training process of the AI processor 140 at a certain time when the ultrasound system 100 is not likely to be used. There may also be an option for selecting specific days, if not every day, when the training may commence at the specified time.

Additionally, there may be an end time associated with any training session where if the training takes more than a certain amount of time, the training session is terminated with appropriate logged messages of where and when the training ended. This may help detect, for example, errors in the training process, or allow the ultrasound system 100 to be used when the training is taking too long. Various embodiments may also save all information present at the time of termination so that the training session may continue at a later time, either automatically when the training start time is available or upon being continued manually.

At block 208, verification setup may be specified. Various different verification steps may be selected, including, for example, verifying specific test data with the newly trained AI processor 140. Verification may comprise, for example, automatically comparing the test images generated by the newly trained AI processor 140 with reference images stored in, for example, the archive 138. The reference images may have been loaded at the factory prior to shipping the ultrasound system 100 or loaded when software was installed on the ultrasound system 100. The reference images may also be updated by adding more images and/or replacing some images. The operator may also select, for example, a percentage of the test images that need to match the reference images to pass verification, as well as set a percentage correlation of a test image with a reference image in order to have the test image pass as a good image.

At block 210, the operator may select other ultrasound systems (as shown in FIG. 1B) to share the corrected images with so that the other ultrasound systems can also be trained using the corrected images. The operator may further enable receiving corrected images from one or more other ultrasound systems. The other ultrasound system(s) may be identified by a pre-assigned name/number, IP address, etc. A group of ultrasound systems may also be selected by, for example, selecting a network accessible to the ultrasound system 100. The network may be, for example, a local network, a local area network, a wide area network, or any specified network that may be displayed to the operator on, for example, the display system 134.

At block 212, the operator may specify whether the ultrasound system 100 may share its corrected images with a central server such as, for example, the central server 192. When the corrected images are to be shared with the central server, there may be further options on when the corrected images are transmitted. Similarly, there may be an option to allow trained models and/or training data to be received from the central server, and when to receive the trained model and/or training data.

It should be noted that the number of options described that may be available to various embodiments of the disclosure is limited for the sake of brevity. Various embodiments of the disclosure may include other options. For example, even when auto mode is selected, various prompts may be provided to the operator as to whether the corrected image should be used for future training. There may also be an option present that allows the operator to specify “do not ask again” or “use the same answer for future situations” when a similar situation occurs. This may prevent the operator from having to repeat the same answer for different corrected images. Accordingly, this may allow the auto mode to bypass interacting with the operator. Various embodiments may allow different levels of interaction with the operator.

Various embodiments may not have some options. For example, in some embodiments the ultrasound system 100 may always share its corrected images with a central server, and may always be allowed to receive trained model and/or training data from the central server.

While an example flow diagram 200 was shown in FIG. 2, it may be noted that various blocks may be added or subtracted, or some blocks may be performed in a different order.

FIG. 3 is an exemplary flow diagram for providing corrected images for continuous training, in accordance with various embodiments. Referring to FIG. 3, there is shown a flow diagram 300 comprising blocks 302 to 312. At block 302, the ultrasound system 100 may display a scanned image via, for example, the display system 134. A processor, such as, for example, the signal processor 132 or the AI processor 140 (or another processor) may detect whether an operator made a correction to the displayed image. If there is no correction detected, then the next step may be to go back to block 302 to wait for the next scanned image to be displayed.

If there was a change made to the displayed image, then, for example, the AI processor 140 may determine at block 306 whether the continuous training mode is set to auto mode or manual mode. If auto mode is set, then the AI processor 140 may anonymize the corrected image and save the anonymized image to, for example, the archive 138. The next step may then be to block 302 to wait for the next image.

If continuous training is not set to auto mode, then the next step may proceed to block 310. At block 310, a prompt may be provided for the operator. For example, the prompt may ask the operator to determine whether to save the corrected image for continuous training. If the operator agrees to save the corrected image, then the next step may be to proceed to block 308 to anonymize the corrected image and save the anonymized image. If the operator declines to save the corrected image, then the next step may be to proceed to block 302 to wait for the next scanned image to be displayed.

An embodiment of the disclosure may also provide an option to never ask if a corrected image should be saved. This may correspond to, for example, turning off the continuous training mode.

An embodiment may also allow weighting different anonymized images with different weights. For example, the weight may depend on the amount of correction needed. Additionally, the anonymized images may also be weighted based on an experience of the operator. Accordingly, an operator may input his/her experience level. There may also be automatic tracking of the operator(s) using the ultrasound system 100, and, for example, the AI processor 140 may update number of images associated with that operator. The operator may also select a weight to be applied for an image.

In manual mode, the operator may be prompted to select a weight for the corrected image, where the operator may keep the default weight of, for example, one or enter another weight that is less than one. There may be an option set for the auto mode, for example, that verifies all calculated weight with the operator, or those weights that are less than one, or those weights that are below a certain threshold, etc., where the threshold may also be set by the operator.

FIG. 4 is an exemplary flow diagram for training the AI processor with the corrected images for continuous training, in accordance with various embodiments. Referring to FIG. 4, there is shown a flow diagram 400 comprising blocks 402 to 406. At block 402, the AI processor 140 may determine that it is time to perform continuous training. Then, at block 404, the AI processor 140 may start the training session using the training engine 160 and at least the corrected images that have been anonymized and stored in the archive 138 since the last training session.

After training is completed to generate an updated AI model, verification may start at block 406. Verification may comprise testing the updated AI model on a fixed data set, which may be stored in the archive 138. The data set may have been loaded at the factory prior to shipping the ultrasound system 100 or loaded when software was installed on the ultrasound system 100. The reference images may also be updated by adding more images and/or replacing some images. A new verification score for the updated AI model may then be compared to the old verification score of the previous AI model. If the new verification score is less than the old verification score, then the updated AI model is not used and the previous AI model continues to be used.

The verification score may be based on, for example, a mean absolute accuracy against known distances and detectability of specific items for distance measurement. When the verification score of the new model is equal to or greater than the verification score of the previous AI model, a notice may be provided to the operator of the ultrasound system 100 via, for example, the display system 134. The notice may state that a new AI model is available based on local updates, and provide the operator a choice of selecting the previous AI model for continued use or the new AI model for future use. There may be provided, for example, verification metrics such as mean absolute accuracy, detectability, etc. so that the operator can take that into account in making a selection. In some embodiments, the selection of the AI model to use may be automatically selected based on an algorithm of each metric for the previous AI model with respect to the corresponding metric for the updated AI model.

There may be stored in the archive 138 and/or the memory 142, for example, various ranges for the metrics. Accordingly, these one or more of these ranges may be selected by the operator for use in determining an accuracy of an AI model. There may also be an option to enter specific ranges for metrics, including a specific value rather than a range.

Accordingly, the AI model for the ultrasound system 100 may be updated much more often than if trained model and/or training data was provided periodically by, for example, the central server 192. Various ultrasound systems may also provide anonymized images to, for example, other nearby ultrasound systems for their training.

Regulatory safety process that was applied before the ultrasound system 100 was shipped may also be applied automatically after each training session to ensure that performance of the AI processor 140 has not degraded. However, in some instances, it may be acceptable to have some degradation as long as the accuracy is above a pre-determined threshold.

Additionally, while images were discussed for the sake of convenience, various embodiments of the disclosure may also apply to image loops (cine loops).

Accordingly, it can be seen that various embodiments provide for a method described in the flow diagram 200, 300, and/or 400. The method for continuous training of an artificial intelligence (AI) model for an ultrasound scanner may comprise generating an image of a target using an AI model, detecting, by a processor, a correction of the target image by an operator, and saving one or both of: the corrected image, and the target image and correction data to the target image. The ultrasound scanner may initiate training the AI model using one of: the corrected image, and the target image and correction data for the target image. The target image may be, for example, a cine loop.

The target image is an image of a target of the ultrasound scanner. For example, the target may be a body part of a patient or a device that may be scanned by an ultrasound system. Accordingly, a target image may be defined as a recorded image with annotation, segmentation, classification, etc.

The term “corrected image” as used in this disclosure refers to corrections that may be made regarding the image. For example, while there may be some corrections to the image, it should be understood that “corrected image” also refers to correction of the classification of the image, correction to the segmentation/landmark detection on the image, changes to the annotation of the image, etc. Accordingly, a “corrected image” may refer to changes to the image data, changes to the metadata of the image, and/or changes to other data associated with the image.

The ultrasound scanner may have one or both of: an auto mode that enables the AI model to automatically save a corrected image, and a manual mode where the ultrasound scanner provides a prompt for the operator to enter whether to save the corrected image. When the manual mode is selected, a field is displayed to the operator to enter a weight for the corrected image that is different from a default weight. A “do not ask again” option may be displayed to the operator as an option to be selected.

The method may comprise using, for training the AI model, an anonymized image that is an anonymized one of: the corrected image, and the target image. When the target image is anonymized, the target image may be processed with the correction data for the training.

Various embodiments of the disclosure may share the anonymized image with a local ultrasound scanner, where the local ultrasound scanner is on a same local network as the ultrasound scanner, and an anonymized image is one of: the anonymized corrected image, and the anonymized target image and correction data.

The training may be initiated at a first preset time, and the training may be terminated when a second preset time is reached. The training may comprise verification that includes determining a first verification score, which may be based on, for example, a mean absolute accuracy and detectability using a verification dataset. When the first verification score is greater than a stored verification score for a previous AI model, the trained AI model may be selected to be used by the ultrasound scanner. When the first verification score is less than a stored verification score for a previous AI model, the previous AI model may be selected to be used by the ultrasound scanner.

The ultrasound scanner may receive an external anonymized image from a local ultrasound scanner, where the local ultrasound scanner is on a same local network as the ultrasound scanner. The AI model of the ultrasound scanner may be trained using at least the external anonymized image.

Certain embodiments provide a non-transitory computer readable medium having stored thereon a computer program having at least one code section. The at least one code section is executable by a machine for causing the machine to perform steps described in the flow diagrams 200, 300, and/or 400.

Accordingly, various embodiments of the disclosure may also provide for a non-transitory computer readable medium having stored thereon, a computer program having at least one code section, the at least one code section being executable by a machine for causing the machine to perform steps comprising generating an image of a target using an AI model, and detecting, by a processor, a correction of the target image by an operator. One or both of the following may be saved: the corrected image, and the target image and correction data to the target image. initiating training the AI model of a ultrasound scanner using one of the following: the corrected image, and the target image and correction data for the target image. The ultrasound scanner may have one or both of an auto mode that enables the AI model to automatically save a corrected image, and a manual mode where the ultrasound scanner provides a prompt for the operator to enter whether to save the corrected image.

The non-transitory computer readable medium may comprise using, for training the AI model, an anonymized image that is an anonymized one of: the corrected image and the target image. The training may comprise verification that includes determining a first verification score, which may be based on, for example, at least one of a mean absolute accuracy and detectability using a verification dataset. When the first verification score is greater than a stored verification score for a previous AI model, the trained AI model may be selected to be used by the ultrasound scanner. When the first verification score is less than the stored verification score for the previous AI model, the previous AI model may be selected to be used by the ultrasound scanner.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for facilitating interaction by an ultrasound operator with an artificial intelligence module configured to classify, landmark detect, segment, annotate, identify, and/or track biological and/or artificial structures in ultrasound images.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What are claimed:

1. A method for continuous training of an artificial intelligence (AI) model for an ultrasound scanner, comprising:
generating an image of a target using an AI model;
detecting, by a processor, a correction of the target image by an operator;
saving one or both of:
the corrected image; and
the target image and correction data to the target image; and
initiating, by the ultrasound scanner, training of the AI model using one of:
the corrected image; and
the target image and correction data for the target image.

2. The method of claim 1, wherein the target image is a cine loop.

3. The method of claim 1, wherein the ultrasound scanner has one or both of:
an auto mode that enables the AI model to automatically save a corrected image; and
a manual mode where the ultrasound scanner provides a prompt for the operator to enter whether to save the corrected image.

4. The method of claim 3, wherein when the manual mode is selected, a field is displayed to the operator to enter a weight for the corrected image that is different from a default weight.

5. The method of claim 1, wherein a “do not ask again” option is displayed to the operator as an option to be selected.

6. The method of claim 1, comprising using, for training the AI model, an anonymized image that is an anonymized one of:
the corrected image; and
the target image.

7. The method of claim 6, wherein when the target image is anonymized, the target image is processed with the correction data for the training.

8. The method of claim 6, wherein the anonymized image is shared with a local ultrasound scanner,
wherein:
the local ultrasound scanner is on a same local network as the ultrasound scanner, and
an anonymized image is one of:
the anonymized corrected image, and
the anonymized target image and correction data.

9. The method of claim 1, wherein the training is initiated at a first preset time.

10. The method of claim 9, wherein the training is terminated when a second preset time is reached.

11. The method of claim 1, wherein the training comprises verification that includes determining a first verification score.

12. The method of claim 11, wherein when the first verification score is greater than a stored verification score for a previous AI model, the trained AI model is selected to be used by the ultrasound scanner.

13. The method of claim 11, wherein when the first verification score is less than a stored verification score for a previous AI model, the previous AI model is selected to be used by the ultrasound scanner.

14. The method of claim 1, comprising receiving, by the ultrasound scanner, an external anonymized image from a local ultrasound scanner, wherein the local ultrasound scanner is on a same local network as the ultrasound scanner.

15. The method of claim 14, comprising training the AI model of the ultrasound scanner using at least the external anonymized image.

16. A non-transitory computer readable medium having stored thereon, a computer program having at least one code section, the at least one code section being executable by a machine for causing the machine to perform steps comprising:
generating an image of a target using an AI model;
detecting, by a processor, a correction of the target image by an operator;
saving one or both of:
the corrected image; and
the target image and correction data to the target image; and
initiating, by the ultrasound scanner, training of the AI model using one of:
the corrected image; and
the target image and correction data for the target image.

17. The non-transitory computer readable medium of claim 16, wherein the ultrasound scanner has one or both of:
an auto mode that enables the AI model to automatically save a corrected image; and
a manual mode where the ultrasound scanner provides a prompt for the operator to enter whether to save the corrected image.

18. The non-transitory computer readable medium of claim 16, comprising using, for training the AI model, an anonymized image that is an anonymized one of:
the corrected image; and
the target image.

19. The non-transitory computer readable medium of claim 16, wherein the training comprises verification that includes determining a first verification score.

20. The non-transitory computer readable medium of claim 19, wherein:
when the first verification score is greater than a stored verification score for a previous AI model, the trained AI model is selected to be used by the ultrasound scanner; and
when the first verification score is less than the stored verification score for the previous AI model, the previous AI model is selected to be used by the ultrasound scanner.
